(19) United States
(12) Patent Application Publication
(10) Pub. No.: US 2008/0262432 A1
(43) Pub. Date: Oct. 23, 2008

(54) SYSTEM AND METHOD FOR MANIPULATING A GUIDEWIRE THROUGH A CATHETER

(75) Inventor: Sean Miller, Danvers, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/737,456

(22) Filed: Apr. 19, 2007

(51) Int. Cl. A61M 5/00

(52) U.S. Cl. 604/164.13

(57) ABSTRACT

A system and method for manipulating a guidewire includes a steering tool, a catheter, and a guidewire. The steering tool includes a bore disposed there through. The catheter is disposed within the ore of the steering tool. The guidewire is disposed within a lumen of the catheter such that the catheter is disposed between the steering tool and the guidewire. The steering tool includes a main body and nut. The main body includes a collet portion including circumferentially spaced jaw separated by longitudinally extending slots. The nut is threaded over the collet to close the jaw with sufficient radical pressure to deform the catheter such that the guidewire may be manipulated with the catheter disposed between the guidewire an the steering tool.

### FIELD OF THE INVENTION

The disclosure relates generally to a system and method for manipulating a guidewire extending within the vasculature of a human body.

### BACKGROUND OF THE INVENTION

Guidewires are well known for placing and guiding catheters and other devices in the vasculature of the human body. In a common type of procedure a guidewire is inserted percutaneously into an easily accessed blood vessel. The guidewire then is manipulated to steer the guidewire through the vasculature until the distal end (the end inside the patient) reaches a desired location. In certain applications, the guidewire is advanced into the vasculature simultaneously with a treatment device, such as a filter disposed on a distal portion of the guidewire.

The steerability of the guidewire is important especially when a tortuous path must be navigated to reach the desired location as is commonly encountered when placing a catheter in the coronary arteries. As disclosed in U.S. Pat. No. 4,545,390 to Lary, steering is executed from the proximal portion of the guidewire by rotating, pushing and pulling on the guidewire to cause corresponding movement at the distal tip of the guidewire. The distal tip typically ha a slight bend so that when rotated it can be directed toward a selected one of several vascular branches. Steering the guidewire directly by hand is difficult because of the small diameter and the slippery coating typically found in guidewires.

Steering tools have been developed to alleviate the foregoing problems. Conventional steering tools generally grip the guidewire directly. Thus, when an over-the-wire Catheter is used in a system wherein the guidewire and catheter are advanced Simultaneously, and wherein the catheter has been advanced such that some of the Catheter is still outside of the body, the exposed portion of the guidewire extends from the Proximal end of the catheter at a location well proximal of the insertion point into the body. It is cumbersome for the user to manipulate a steering tool mounted about the guidewire at such a long distance from the insertion point into the body.

### BRIEF SUMMARY OF THE INVENTION

The present disclosure is a system and method of manipulating a guide wire through a catheter or sheath. The system includes a steering tool, a catheter, and a guidewire. The steering tool includes a bore disposed there through. The catheter is disposed within the bore of the steering tool. The guidewire is disposed within a lumen of the catheter such that the catheter is disposed between the steering tool and the guidewire. The steering tool includes a main body and nut. The main body includes a collet portion including circumferentially spaced jaws separated by longitudinally extending slots. The nut is threaded over the collet to close the jaws with sufficient radial pressure to deform the catheter such that the guidewire may be manipulated with the catheter disposed between the guidewire and the steering tool.

The method of manipulating the guidewire includes providing a catheter, a guidewire, and a steering tool. The guidewire is disposed within a lumen of the catheter. The steering tool is mounted over the catheter such that the catheter is disposed between the steering tool and the guidewire. The steering tool is tightened to apply sufficient radial pressure to the catheter and guidewire to manipulate the guidewire.

### BRIEF DESCRIPTION OF DRAWINGS

The foregoing and other features and advantages of the disclosure will be apparent from the following description of the disclosure as illustrated in the accompanying drawings. The accompanying drawings, which are incorporated herein and form a part of the specification, further serve to explain the principles of the disclosure and to enable a person skilled in the pertinent art to make and use the disclosure. The drawings are not to scale.

FIG. 1 illustrates a side view, partly in phantom, of a treatment system for use in a vascular system.

FIG. 2 illustrates a perspective view, partly in phantom, of the steering rod shown in FIG. 1.

FIG. 3 illustrates a cross-sectional view taken along line A-A of FIG. 1.

### DETAILED DESCRIPTION OF THE INVENTION

Specific embodiments of the present disclosure are now described with reference to the figures, where like reference number indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The present disclosure is directed to a device and method for steering a guidewire through the vasculature of a human body, wherein a catheter is disposed between the device and the guidewire. The catheter may be used for performing an angioplasty, artherectomy, dissection, ablation, or other treatment within a patient's vessel.

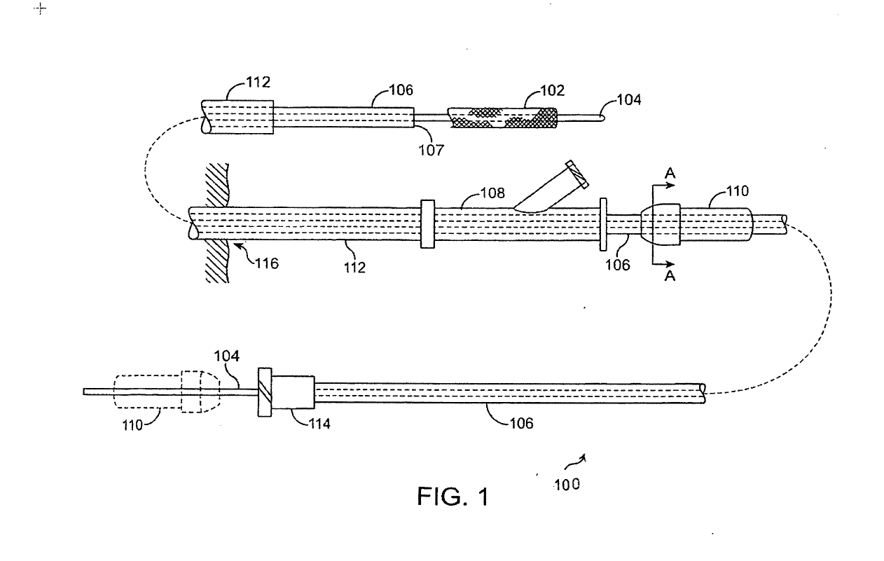

FIG. 1 of the present disclosure illustrates a fragmented side view of a system 100 including a catheter 106, a guidewire 104, and a steering tool 110. In the illustrated example, filter 102 is shown mounted about the distal end of guidewire 104. Catheter 106 may be a delivery and/or retrieval sheath for collapsing filter 102. Alternatively, guidewire 104 may be a conventional guidewire and/or many different devices may be mounted on, or used in conjunction with catheter 106. Other types of catheters (not shown) may be used in system 100 such as, for example, an interventional catheter carrying a balloon, stent, ablating tool, artherectomy tool, etc. Catheter 106 includes a lumen 107 disposed there through.

An optional guiding catheter 112 is shown for directing system 100 to a targeted branch vessel, as will be understood by one of skill the art of cardiovascular catheterization. Coupled to the proximal end of guiding catheter 112 is a luer-type fitting and/or a Tuohy-Borst adapter 108. Guiding catheter 112 is inserted, schematically, into a patient's body at an insertion point 116.

Steering tool 110 is movably or removably mounted around catheter 106 and guidewire 104 at a location adjacent to Tuohy-Borst adapter 108. Such a location advantageously provides a clinician with manual control of system 100 as close as possible to insertion point 116. Steering tool 110 may alternatively be mounted in a more conventional position, directly around guidewire 104 where guidewire 104 exits the proximal end of catheter 106, as illustrated in phantom. The proximal end of catheter 106 may be plain or it may be fitted with hub 114. Hub 114 may be a removable type fitting such that steering tool 110 can be slipped on or off of catheter 106 without removing catheter 106 from the patient. If hub 114 is permanently affixed to catheter 106, then steering tool 110 needs to be pre-loaded onto catheter 106 before system 100 is inserted into guiding catheter 112.

Catheter 106 may be made of any conventional catheter material, such as polyethylene, polyamide, or polyurethane. It may be desirable for a distal portion of catheter 106 to be formed of conventional slippery materials such as polyethylene, polyamide, or fluoropolymer for ease of tracking over the guidewire. It may also be desirable for a proximal portion of catheter 106 to be made of less slippery materials such as elastomer, nylon, polyimide, polyethylene terephthalate (PET), polyurethane, polyvinyl chloride, or blends or copolymers of the above for improved gripping with the guidewire, as explained below. Catheter lumen 107 is typically larger in diameter than guidewire 104 to provide clearance that enhances relative sliding movement between guidewire 104 and catheter 106. At least a proximal portion of catheter 106 is sufficiently radially compressible such that steering tool 110 can pinch catheter 106 into gripping engagement with guidewire 104, as described below.

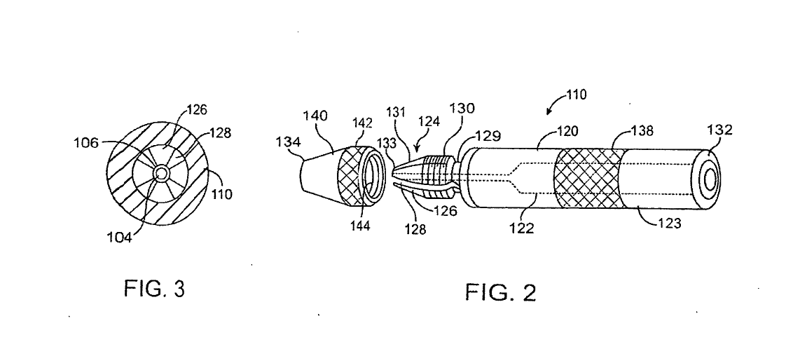

FIG. 2 illustrates steering tool 110. Steering tool 110 includes a body 120 and a nut 140 threaded onto an end of the body. Body 120 includes a proximal end 132 and a distal end 133. Body 120 includes a generally cylindrical portion 123 at a proximal portion and an integral collet portion 124 at a distal portion. Collet 124 is tapered distally and includes four circumferentially spaced jaws 126 separated by radial, longitudinally extending slots 128. As would be understood by those of ordinary skill in the art, the collet jaws may number more or less than the four jaws 126 shown in the present example. A reduced-diameter neck 129 may ex tend between collet 124 and cylindrical portion 123 to reduce the bending force required to close jaws 126.

Body 120 includes a longitudinally extending bore 122 that may narrow from a wider diameter at proximal end 132 to a narrower diameter at distal end 133. Collet 124 includes an externally-threaded portion 130 and a distally tapering, generally conical portion 131. Nut 140 is shaped and sized to fit over collet 124 and includes internal threads to engage externally-threaded portion 130. Nut 140 also has an internal conical surface 144 that is drawn against generally conical portion 131 to close collet jaws 126 when nut 140 is tightened about collet 125. Nut 140 is open at distal end 134. Bore 122, jaws 126 and slots 128 are sized and spaced such that catheter 106 may be slidably disposed within bore 122 when jaws 126 are open. Steering tool 110 may accommodate catheters having outer diameters, for example, of approximately 0.035 inches. When nut 140 is tightened about collet 124, jaws 126 close to apply radial pressure on catheter 106 and guidewire 104 disposed through catheter 106. Further, jaws 126 and slots 128 are sized and spaced such that steering tool 110 may directly grip conventional 0.014 inch diameter guidewires without catheter 106 interposed between tool 110 and guidewire 104. Another exemplary system in accordance with the disclosure may comprise a 0.035 inch diameter guidewire and an associated catheter having an outer diameter of about 0.070 inches. Thus, steering tool 110 may be adapted for jaws 126 to grip a range of diameters of various devices inserted there through. An outer surface of body 120 may include a knurled portion 128 and an outer surface of nut 140 may also include a knurled portion 142 for enhanced grip by the user.

Body 120 and nut 140 preferably may be formed of brass or other metal such as aluminum, stainless steel or alloys of brass, aluminum or stainless steel. Hard plastics such as acetal, acrylonitrile butadiene styrene (ABS), polyamide, polyvinylchloride, acrylic, polycarbonate or polystyrene may also be used. Jaws 126 may also be formed of brass or other metal such as hard-coat anodized aluminum, stainless steel, nitinol, or alloys of brass, aluminum or stainless steel.

In one embodiment of practicing the disclosed method, system 100 is assembled by inserting guidewire 104 through catheter 106 and steering tool 110 over guidewire 104 and catheter 106. Thus, catheter 106 is disposed between steering tool 110 and guidewire 104. System 100 is then inserted into the vasculature of the patient's body, optionally through guiding catheter 112. Nut 140 is tightened on body 120 to close jaws 128 around catheter 106. Jaws 128 are closed with sufficient force to deform catheter 106 into gripping engagement with guidewire 104 such that guidewire 104, together with catheter 106, may be steered or otherwise manipulated by steering tool 110, similar to steering tool 110 or conventional steering tools mounted directly onto a guidewire. Nut 140 may be loosened on body 120 to move, remove or relocate steering tool 110 along catheter 106 and/or guidewire 104 after advancing catheter 106 into the vasculature.

While various embodiments of the present disclosure have been described above, it should be understood that they have been presented by way of illustration and example only, and not limitation. It will be apparent to persons skilled in the relevant art that various changes in form and detail can be made therein without departing from the spirit and scope of the disclosure. Thus, the breadth and scope of the present disclosure should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A method of manipulating a guidewire comprising the steps of:
providing a catheter including a lumen disposed there through;
providing a guidewire disposed through the lumen, wherein said guidewire is movable with respect to the catheter;
providing a steering tool;
mounting the steering tool over the catheter such that the catheter is disposed between the steering tool and the guidewire;
tightening the steering tool to apply sufficient radial pressure to pinch the catheter into gripping engagement with the guidewire to manipulate the catheter and the guidewire together.

2. (canceled)

3. The method of claim 21, wherein the body includes a plurality of jaws with longitudinal slots disposed between the jaws.

4. The method of claim 21, wherein the body includes a collet portion with threads disposed on an outside surface thereof, and wherein the nut includes threads on an inside surface thereof, and wherein the step of tightening the steering tool comprises threading the nut onto the collet portion.

5. The method of claim 1, wherein the step of tightening the steering tool applies sufficient pressure to the catheter to deform the catheter.

6. The method of claim 1, wherein the catheter is formed from a material selected from the group consisting of polyethylene, polyamide, and polyurethane.

7. The method of claim 1, wherein a distal portion of the catheter is made from a material selected from the group consisting of polyethylene, polyamide, and fluoropolymer, and a proximal portion of the catheter is made from a material selected from the group consisting of elastomer, nylon, polyimide, polyethylene terephthalate, polyurethane, polyvinyl chloride, and blends or copolymers thereof.

8. The method of claim 1, wherein the steering tool is formed from a material selected from the group consisting of brass, aluminum, stainless steel, alloys of brass, aluminum or stainless steel, acetal, acrylonitrile butadiene styrene, polyamide, polyvinylchloride, acrylic, polycarbonate, and polystyrene.

9. The method of claim 3, wherein the jaws are made from a material selected from the group consisting of brass hard-coat anodized aluminum, stainless steel, nitinol, and alloys of brass, aluminum or stainless steel.

10. The method of claim 1, wherein the catheter includes a distal portion made of a first material and a proximal portion made of a second material, wherein the first material is more slippery than the second material.

11. A system for delivering a device to a treatment region within a vessel comprising:
a steering tool including a bore disposed there through;
a catheter disposed within the bore of the steering tool, the catheter including a lumen disposed there through; and
a guidewire disposed through the lumen such that the catheter is disposed between the steering tool and the guidewire, wherein the guidewire is movable with respect to the catheter;
wherein the steering tool is capable of being tightened to apply sufficient radial pressure to pinch the catheter into gripping engagement with the guidewire to manipulate the catheter and guidewire together.

12. (canceled)

13. The system of claim 22, wherein the body includes a plurality of jaws with longitudinal slots disposed between the jaws.

14. The system of claim 22, wherein the body includes a collet portion with threads disposed on an outside surface thereof, and wherein the nut includes threads on an inside surface thereof such that the nut is capable of being threaded onto the collet portion.

15. The system of claim 11, wherein the steering tool is capable of being tightened to apply sufficient radial pressure to the catheter to deform the catheter.

16. The system of claim 11, wherein the catheter is formed from a material selected from the group consisting of polyethylene, polyamide, and polyurethane.

17. The system of claim 11, wherein a distal portion of the catheter is made from a material selected from the group consisting of polyethylene, polyamide, and fluoropolymer, and a proximal portion of the catheter is made from a material selected from the group consisting of elastomer, nylon, polyimide, polyethylene terephthalate, polyurethane, polyvinyl chloride, and blends or copolymers thereof.

18. The system of claim 11, wherein the steering tool is formed from a material selected from the group consisting of brass, aluminum, stainless steel, alloys of brass, aluminum or stainless steel, acetal, acrylonitrile butadiene styrene, polyamide, polyvinylchloride, acrylic, polycarbonate, and polystyrene.

19. The system of claim 13, wherein the jaws are made from a material selected from the group consisting of brass, hard-coat anodized aluminum, stainless steel, nitinol, and alloys of brass, aluminum or stainless steel.

20. The system of claim 11, wherein the catheter includes a distal portion made of a first material and a proximal portion made of a second material, wherein the first material is more slippery than the second material.

21. The method of claim 1, wherein the steering tool comprises a body and a nut, and wherein the step of tightening the steering tool comprises tightening the nut on the body.

22. The system of claim 1, wherein the steering tool comprises a body and a nut.
